SEQUENCE LISTING (16 sequences)

SEQ ID NO: 1 - DNA, 8500 bp, Homo sapiens
tcagagatacaaatacagtgtaataccaaaatctcagtacattttcaccatgaaaacaaataggaaaagcacaagactgtcaggagtttccttccttttccgcagtagctgacacaactgcttcaaagcaatgattttttttctttagggactgtatcattgacaggttttacaatttcatttaacactgtgctttgttgagaaataaccacttatttctattttaagggagtcatcatattggaattaaaacacatgtttacaatacatattggcacaatatgaaaaataaacactttttgaattttcaaccccaaacaattgttattaaataaataattattcctcatagtgcatgtcttaatttacctgtcattgcccattgacacaaatgaagctgaataaataatgttaggtagtttattaacgtcttctttcataattctgcattgcactcctttcataagccactgaaaacaaagaagagataaagtgttttttaagtgaattcactgagggagttacacaactaaagttcagggcatatagtaatttattttaaactcataatgacattttccctctacagaagtatttctaagaatgttatctgcacatcttccaatcctgcctcagtctctattctgattggaaatgatctaaccacagaactcggtttcattcatcattcatcatatacttggtgcccccaaagcatatttccacaatatattgcagtgttgattctcagcagctttctcaagcagtggcagatacccatagtgaattctagaaaggacaatgcctaaaataaccaggaatactagtaggataaaaaccaaatgtgggacttttattctagcagagaccatgggccaaatctagaataccatttgttcttgtaaagaggtttataggaatacagacacactcattcatttacatattgtctatggctgctttggcctaaaataacagatttgggtagttgcaacagagattacatggctcacaaagcctaaaatatttataatctgtttcttttcaggaaaagtttgctgatctttgttcaaggggcatctaattgtgtaggggtctcttcctaccttgtggatacctcccagtgatggactgccctcaagactaaaaacagcccatttaccataggtaagtcagttgagccatctaaataccatactgtttaacttttcactagcaccttaatggcctgtgaagcattaattattgcttccaaaggtatttaacatatagctagcaagtaatttaaatacattgattatctggacaaaaaaatgccaaaattgtatattatattatattatatttattatatataatttcaaagggatctgtaaccacaaaggggtaaaaatcagactgttgcaataaaagtattttgggatatttttcatccttaacgaataccttatagacttagacgtatggtatagtggaactactatgctttttgaaattcagtatggcctgtataaaatactgaatgtaaacagaaagagtagaaagatctgtgaaggatctgtaggaataataagtgctactctaaacagtttcctcagtgagaagatttgctgacagagaagacaggtaaaggcaccagtattatcacttccctcttttaatactggtcttaaacacccagctatcctttaagtgcggttaacaataagctgcatattttacatcaattaagtatgcacttgtgaggcagaacagtctgagccaacactggacaggcgtgagggaaggaaacagtttatttctcattctggtttgattatattaggaacaggatgaatggcccaagttcagtctctgacaagctttaggtaacatgactctattcctcagagaaattagatgaaagctaatgtaaaccaactaaagacagacagtgatttagctggaacataatgtcctcttacatggaagcagagtttcttacctggtctttgaaccctctgaagcctgatataaatttttgagtgtatgagctgatgagggtttctctgggaaagggcatctatctagatttatcagattgtcaaaggggtccctgaccatgagtgggtaaagatcagagggctatagattaaggctatgataaccttgcctcaatgaggtacataaaaagatccctgattgactgtggatcatatcacatttttttccacatttcacctatttatatttccttaactaaaatggaggaacaaagatttagttataaattgatcctgattcactacagtgatttttatcactacttaaaattatgttattattttaagtacatttaattgtatttaatctgttctaacttgtacaatatctacatttgtaaagttaagcatgttctgaaacctatatcaattctggaaatggaaagttgatgactaatatgattatcaagaaaactacaattaagtgaggactagtatgtattggtacaaataaaagccattgtggtttaaatttatatttcatttccaaaagtgggaagataatacatattaataaaacaattacaagtttttcattaaggcacattgataatttcttacacttttacacttcggcagcccaaaatgactggtgagaagatgcaaaggtagtgtctaaatagaaagtggggacccaataagaacactaggaagtcctgtggacttcaccaagaacttcaaattccagatttattggaaattttgtatcaaaaggtcaattatgaaacacttagaacttttcaattatatgaatatattctctggcttctgccgtagcccagattgtgttaactttttttttctattgcgcgttttgatttttgccccacagaaagtggttttatttaatgtacccaagtcttacatttcacaataaagaaaaaaatctccattaaaaaacactaggataattatttttcctaggatccagataattaaggattaagccagcacatcttatagattgtctagaaactggaaatattctaaatgggtaagacagctgctgattactttataattaataacgtatttagtattttaattctattttcactgaaggcaaaatagaatttgaatagcttttccaagaagccctagaggtaagagctataaggtcttaacaaaaagtgagcttctattaaaatgatctaggggttatttacattgaaatcagtgtgatgaatgagttaactaataatggttttcagatatatgtacgtggtatttacaaagaagatggaaattgaagagaaggaaacctgcagcctttatatcatacgaaaaagttgttcagaattaaaatgagaattgtggcatcctttctctctaaggagtttatagccagaaccaatttataacactcaaggatatttaattgtgttcctttttgaaagtactggaataaaccgtatcacttttgtcctctccaacatccatcaggatgtttagattttgaatttctacaacttcataaaaagtgattaatttgcataaagaatttgttttgtgaatttatttttgtattggaagatcgccaaatgtaatgtcttgtaaaactttcaaattactttgataacaagtgaaaatcaccctcattcttccctgcactccccactcccccttagttcagtaaggttaaacacggcttatttaacagatctataggaaagaatatagcactaacccaaagattcaggttggtaatgattaaatggggcagacactggtaagaatggtgattggagagaaatagacagatactgtggtagtattagcttaaccagctataaatccatttgtggttacaagtacaacaattaaatttttccaccataatactcatttttatatctaccactcctttttaatattttaaaactatgtcttagaaaaaatagaaatagatttgagatactgtctattttcagtcataatttcatacataccagaatttaaagctcttttgcccattagtccaacaaaggaatctgttttatgtcctggaaaaatacatttaggggtattttaatatgtatatctttgaggaaacaaactattataaccagaacaaagcaagcttctgaatatatgtctgtataataaatatctatctacttcaagttttggtgagactgaagaaataaagattccttaaaagttttataaattacactaaaacaatacaatgaaaccacttttcttaattctatatttaatattagcaagcactctctattcatatattttatctgttatgtaagggtctaaaatattttatataaaatattgttatttgagctaggtttgattttttaggaagtcaatttcacatttgtgtgagagtgaaatatccaagcataattccttgtgttattaataagttgtacttctcagcaagtagaccttaattaaaccagattcctttccttgatgattataatcttctaaaaggtcactgtttttattttattggatattaaaagatactcctttcagaaaagtttattagttttcacatgaacagagtacattttttattacatagaatttttatatataattctcttcaaagtcaaatccttagaaagtaaatatgaatactatttatttttcaaagatgaactctttggaaaggttactattttaatgctagtgagagaatttctcttggtttttattttaatagttgagaatagagctgctacgttaatgtggcctgctctgccacccaatgaaccaaatccatgacagggtacaataggtgagtggacaatacgatgaagatagaccctttccacttcttgtcaactccggttccattaactgagaaacaaatttaatgaccttcacataccacaattttgccatctttgctgccctaagaaatgttttactgatcgagttttcccattcaattaggggattaaaaaactgcatctctaccgtcttcatttttagtatggattctctactcatgttcactcactcattccatttaattggctgctggatgaccctaataaatgacatcattattggaccaagcatactaaaatgagttaggagatgcctataaatgcttgtgggggtttcgtgtttgtttgtttgtttgtttacagtgaaagtaagcttagggggtgggtggctgggaagaaatatgtatggtagatttctaatcctaggattgaaattcttctctaaaactaacaattgaagggacttaaaagacatttttttcttttttaatataatgcatctctttgctaagtacaatatgtctaaacaaccatattctggatgtaatatgaatctcccccattcttatattgtcttcattaaacatattaagccctagagtcatgacagaaaataaggtaagataaataaagtgaaattcaatataattttacatttaaatttgataaatgtcaaaataattttgaacttacttttgtgagagatctggccatgtcctagaagaaagataaaattagcaaacacatatagatttgtttttaaagatgctacattatatcttttataaaataatttgattctcaaaatatgtaacattttctatctatatattatcctgaggaataaagcagaaccataaatgctaaacaaattattaatcataaaaactaaatatttaaacaaacaaatggaatttttattaagtgccttctatgggattcaaacctaggactatgagacttctgaggcctcttattaactataagactgtaacacatcccaccttcaaggctggccacccactgagagagagagagaaagagagagagagagactgagagagagtgacagagagagagagacagagagagtgacagagagagagagaaaccgagagagagtgcgcaaatccatctctttattaaacaggcatgacccagatcttgtatttcagcccataaaagattttatttcactttcctacatgtgctttcacaatagtaaaataaagatttataggaatgtttaccataaagagcctttaacagggccacttgtttttcaattgaggaatctgacaagagacgaattgtcattaactacatgttccactgaccgtgcgatgaattcaaggaaacgatatttctatcccaagacaaatgtactctcacctcagggaatgctataaaatcctcttccacatgcacttggatcatccgcagtactacccctggaggcccgtgccttccctttgtcagtttggatctttagtcaagggagcgcttcactcgaatacagtgaggcatgcgaaccagatctttccctccccccgcgggcccgtgcgtgccttagtgcggtgtgggccaatgtcgcgcctggggaattacacccctcttggagtggaaatctctattcagagcgagagaccctggggtactccaggaaggagtgagcagacagggcgggctgcccaagacacctagggacggtcgcctatctcaccagcatcccgtttgcccattaatccaaagaactgctgaggcttgggtctccgggcgattctctgcagaagatgctcaaagggctccggcagttcctcctgggagacaaaagcacggacaaagggcgtgtaaggcaaccagagcgaggcacccagcaacccgagcccttccccccgctgtactccggccccgaggcgtggggaggcggccttccccacgctcgggctgagcggggtccctcccgaggcgctgcctctctgcgcccctccccgccgaactcgcgtcccgccccccgcaagcctgccagggcgagttagcagctggctcggcgtcttcaagcttcgagcgctttccgctctttcctcccagcccggggaccttccctcatctgctgcgccgccacctgactttgtcttgtctcggtgccggctttgtagagtcctcaccgccctaaatgcacacaggtgtgtgggatctggggctggggctccggtggggaagggtgacaacgctttacagacgactttcaccgtttttccgaggcgcgctccccagcgcctgtagcctacgagtttctaaagggagacttcaagctacatcctcaggtccgcaaaacaaacgtgaaggcaggaaattaacttccctccagcctccggccctccaatccccctgcagctcgcgcgtgtgcccgctgctgcccgcgacccaggcgccctcctctgcccgtgcccagacgctagggcagtgcagctggaccggagcccccgaaatcccaggtcctgggggatgaaatcttacgagatttcccgccttgtgaggatctcgggctcatccgggagggcatccctactgcagggagttcctgggccggacggtgcagccctcgggaagaaacttgaatcgtgggcacaaaatcccgtggggaacccgggaaatagtgcccctttccatcctctcgcacgcaaccgctgcctctttccgtggtggcggtgcgtgccacgttaactagggtgctgttgcgtgggaaggtgacagctcccgagcccaggaagaagggtgcgggccgtcctgggaaggggcctcaccttgatctggtcgctgtcgtaccagtcggaccagtaattcagatcatcattggctcctatttcttctgcaaacagctgagtggagacaagaaaaaagactgccaaggccacgaggattttcatgttggatttctgggaaggggtaggaggagaagacaccaaagagagaaataatgtatctattaggggtggttatccaagagttaaagcaacagaattttctgccccacaacggatgaaccaagatcaaaaacataaggactgaagtctcacctgtaccggttaggcgagagaatcgcgtcccaaagattctccaaccctcttctccctgaatcccaactcccccagccccctaacccaccaacccacaccttcaaaccaggaaactctgcaggtggaagacaaagaggggcgttggcgtttgggaagccagtctccccactgtccctcgccgcagcggcgcccccagcgcggccacctcggacagatgcggggcggggtgacccgggcagccgcggccagcaccgcgcgggcacttactgcgacggacagtcccgcggggtgctgagtttctctggttcctccgagcgcacgctggtcgctccgcactctcggtagctgccgccgggaaggaggtccgagcgcgctctttcgcctgctcagtgccttgcggtatttatcccagcctccttgagcctccagacccacgtgacattctccccacgcgactttctgctcaatatttaattagccgcctcctctcctttcgcttgcgtgattgagagtttccgagacggtaactcgtcgatgcccataacatctggacccaattgggttctaaatgacgcaattttaggaaaatcgaggtctcgccatccaatccggagcggcc

SEQ ID NO: 2 - DNA, 24 bp, Homo sapiens
catgtttacaatacatattggcac

SEQ ID NO: 3 - DNA, 17 bp, Homo sapiens
gtatatgatgaatgatg

SEQ ID NO: 4 - DNA, 19 bp, Homo sapiens
caccctcattcttccctgc

SEQ ID NO: 5 - DNA, 21 bp, Homo sapiens
cttcagtctcaccaaaacttg

SEQ ID NO: 6 - DNA, 20 bp, Homo sapiens
gtgcccctttccatcctctc

SEQ ID NO: 7 - DNA, 20 bp, Homo sapiens
ggtgtgggttggtgggttag

SEQ ID NO: 8 - DNA, 19 bp, Homo sapiens
ccggtacaggtgagacttt

SEQ ID NO: 9 - DNA, 19 bp, Homo sapiens
ccggtacaggtgagacttc

SEQ ID NO: 10 - DNA, 19 bp, Homo sapiens
caacggatgaaccaagatc

SEQ ID NO: 11 - DNA, 26 bp, Homo sapiens
agagaaatagacagatactgtggtag

SEQ ID NO: 12 - DNA, 26 bp, Homo sapiens
agagaaatagacagatactgtggtaa

SEQ ID NO: 13 - DNA, 22 bp, Homo sapiens
atggatttatagctggttaagc

SEQ ID NO: 14 - DNA, 26 bp, Homo sapiens
cagatctataggaaagaatatagcac

SEQ ID NO: 15 - DNA, 26 bp, Homo sapiens
cagatctataggaaagaatatagcat

SEQ ID NO: 16 - DNA, 26 bp, Homo sapiens
ccatttaatcattaccaacctgaatc

(19) United States
(12) Patent Application Publication
(10) Pub. No.: US 2003/0158187 A1
(43) Pub. Date: Aug. 21, 2003

(54) Genetic polymorphisms in the preprotachy kinin gene

(76) Inventors: Dorothee Foernzler, Lenzburg (CH); Lara Hashimoto, Basle (CH); Jia Li, Union City, CA (US); Eric Luedin, Liestal (CH); Andrew Sleight, Riedisheim (FR); Pierre Vankan, Basle (CH)

Correspondence Address: HOFFMANN-LA ROCHE INC., PATENT LAW DEPARTMENT, 340 KINGSLAND STREET, NUTLEY, NJ, 07110

(21) Appl. No.: 10/354,693

(22) Filed: Jan. 30, 2003

(30) Foreign Application Priority Data
Jan. 31, 2002 (EP) 02001937.8

(51) Int. Cl. C12Q 1/68; A61K 31/44; A61K 31/541; A61K 31/5377

(52) U.S. Cl. 514/227.5; 435/6; 514/235.5; 514/357

(57) ABSTRACT

The present invention relates to a method for correlating single nucleotide polymorphisms in the preprotachykinin (NKNA) gene with the efficacy and compatibility of a pharmaceutically active compound administered to a human being. The invention further relates to a method for determining the efficacy and compatibility of a pharmaceutically active compound administered to a human being which method comprises determining at least one single nucleotide polymorphism in the NKNA gene. Said methods are based on determining specific single nucleotide polymorphisms in the NKNA gene and determining the efficacy and compatibility of a pharmaceutically active compound in the human by reference to polymorphism in NKNA. The invention further relates to isolated nucleic acids comprising within their sequence the polymorphisms as defined herein, to nucleic acid primers and oligonucleotide probes capable of hybridizing to such nucleic acids and to a diagnostic kit comprising one or more of such primers and probes for detecting a polymorphism in the NKNA gene, to a pharmaceutical pack comprising NK-1 receptor antagonists and instructions for administration of the drug to human beings tested for the polymorphisms as well as to a computer readable medium with the stored sequence information for the polymorphisms in the NKNA gene.

### BACKGROUND OF THE INVENTION

The present invention relates to a method for correlating single nucleotide polymorphisms in the preprotachykinin (NKNA) gene with the efficacy and compatibility of a pharmaceutically active compound administered to a human being. The invention further relates to a method for determining the efficacy and compatibility of a pharmaceutically active compound administered to a human being which method comprises determining at least one single nucleotide polymorphism in the NKNA gene. Said methods are based on determining specific single nucleotide polymorphisms in the NKNA gene and determining the efficacy and compatibility of a pharmaceutically active compound in the human by reference to polymorphism in NKNA. The invention further relates to isolated nucleic acids comprising within their sequence the polymorphisms as defined herein, to nucleic acid primers and oligonucleotide probes capable of hybridizing to such nucleic acids and to diagnostic kits comprising one or more of such primers and probes for detecting a polymorphism in the NKNA gene, to a pharmaceutical pack comprising NK-1 receptor antagonists and instructions for administration of the drug to human beings tested for the polymorphisms as well as to a computer readable medium with the stored sequence information for the polymorphisms in the NKNA gene.

Pharmacogenetics is an approach to use the knowledge of polymorphisms to study the role of genetic variation among individuals in variation to drug response, a variation that often results from individual differences in drug metabolism. Pharmacogenetics helps to identify patients most suited to therapy with particular pharmaceutical agents. This approach can be used in pharmaceutical research to assist the drug selection process. Details on pharmacogenetics and other uses of polymorphism detection can be found in Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Moreover, polymorphisms are implicated in over 2000 human pathological syndromes resulting from DNA insertions, deletions, duplications and nucleotide substitutions. Finding genetic polymorphisms in individuals and following these variations in families provides a means to confirm clinical diagnoses and to diagnose both predispositions and disease states in carriers, as well as preclinical and subclinical affected individuals. Counseling based upon accurate diagnoses allows patients to make informed decisions about potential parenting, ongoing pregnancy, and early intervention in affected individuals.

Polymorphisms associated with pathological syndromes are highly variable and, consequently, can be difficult to identify. Because multiple alleles within genes are common, one must distinguish disease-related alleles from neutral (non-disease-related) polymorphisms. Most alleles are neutral polymorphisms that produce indistinguishable, normally active gene products or express normally variable characteristics like eye color. In contrast, some polymorphic alleles are associated with clinical diseases such as sickle cell anemia. Moreover, the structure of disease-related polymorphisms are highly variable and may result from a single point mutation such as occurs in sickle cell anemia, or from the expansion of nucleotide repeats as occurs in fragile X syndrome and Huntington's chorea.

Neurokinin-1 (NK-1) or substance P is a naturally occurring undecapeptide belonging to the tachykinin family of peptides, the latter being so-named because of their prompt contractile action on extravascular smooth muscle tissue. The 3 known tachykinins in man are encoded by 2 genes, NKNA and NKNB. The NKNA gene encodes a precursor containing both substance P and neurokinin A, while the NKNB gene encodes a precursor containing only neurokinin B. (Neurokinin A was formerly known as substance K). Using probes derived from the cloned human genes and a panel of rodent-human somatic cell hybrids, Bonner et al. (Cytogenet. Cell Genet., 1987, 46, 584) assigned the NKNA gene to 7q21-q22 and the NKNB gene to 12q13-q21. Molecular characterization of the tachykinins show that they arise from common precursor molecules known as preprotachykinins by proteolytic processing. Three forms of message (alpha, beta, and gamma) arise by alternative splicing events (Proc. Nat. Acad. Sci., 1987, 84, 881-885). The beta and gamma forms of preprotachykinins encode both substance P and neurokinin A, while the alpha form contains only the substance P sequence.

The neuropeptide receptor for substance P (NK-1 receptor) is a member of the superfamily of G protein-coupled receptors. It is widely distributed throughout the mammalian nervous system (especially brain and spinal ganglia), the circulatory system and peripheral tissues (especially the duodenum and jejunum) and is involved in regulating a number of diverse biological processes.

The central and peripheral actions of the mammalian tachykinin substance P have been associated with numerous inflammatory conditions including migraine, rheumatoid arthritis, asthma, and inflammatory bowel disease as well as mediation of the emetic reflex and the modulation of central nervous system (CNS) disorders such as Parkinson's disease (Neurosci. Res., 1996, 7, 187-214), anxiety (Can. J. Phys., 1997, 75, 612-621) and depression (Science, 1998, 281, 1640-1645).

Evidence for the usefulness of NK-1 receptor antagonists in pain, headache, especially migraine, Alzheimer's disease, multiple sclerosis, attenuation of morphine withdrawal, cardiovascular changes, oedema, such as oedema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases has been reviewed in “Tachykinin Receptor and Tachykinin Receptor Antagonists”, J. Auton. Pharmacol., 1993, 13, 23-93.

Furthermore, NK-1 receptor antagonists are being developed for the treatment of a number of physiological disorders associated with an excess or imbalance of tachykinin, in particular substance P. Examples of conditions in which substance P has been implicated include disorders of the central nervous system such as anxiety, depression and psychosis (WO 95/16679, WO 95/18124 and WO 95/23798).

The NK-1 receptor antagonists are further useful for the treatment of motion sickness and for treatment induced vomiting.

In addition, in The New England Journal of Medicine, 1999, 340(3), 190-195, the reduction of cisplatin-induced emesis by a selective NK-1 receptor antagonist has been described.

The usefulness of NK-1 receptor antagonists for the treatment of certain forms of urinary incontinence is further described in Neuropeptides, 1998, 32(1), 1-49, and Eur. J. Pharmacol., 1999, 383(3), 297-303.

Furthermore, U.S. Pat. No. 5,972,938 describes a method for treating a psychoimmunologic or a psychosomatic disorder by administration of a tachykinin receptor antagonist, such as NK-1 receptor antagonist.

Life Sci., 2000, 67(9), 985-1001, describes, that astrocytes express functional receptors to numerous neurotransmitters including substance P, which is an important stimulus for reactive astrocytes in CNS development, infection and injury. In brain tumors malignant glial cells originating from astrocytes are triggered by tachykinins via NK-1 receptors to release soluble mediators and to increase their proliferative rate. Therefore, selective NK-1 receptor antagonists may be useful as a therapeutic approach to treat malignant gliomas in the treatment of cancer.

In Nature (London), 2000, 405(6783), 180-183, it is described that mice with a genetic disruption of NK-1 receptor show a loss of the rewarding properties of morphine. Consequently, NK-1 receptor antagonists maybe useful in the treatment of withdrawal symptoms of addictive drugs such as opiates and nicotine and reduction of their abuse/craving.

NK-1 receptor antagonists have been reported to have also a beneficial effect in the therapy of traumatic brain injury (oral disclosure by Prof. Nimmo at the International Tachykinin Conference 2000 in La Grande Motte, France, Oct. 17-20, 2000 with the title “Neurokinin 1 (NK-1) Receptor Antagonists Improve the Neurological Outcome Following Traumatic Brain Injury” (Authors: A. J. Nimmo, C. J. Bennett, X.Hu, I. Cernak, R. Vink).

Another indication for treatment with NK-1 antagonists is benign prostatic hyperplasia (BPH), which can be progressive and lead to urinary retention, infections, bladder calculi and renal failure and has been reported in EP 01109853.0.

Clinical trials have shown that patient response to treatment with pharmaceuticals, e.g. NK-1 receptor antagonists, is often heterogeneous. Thus there is a need for improved approaches to pharmaceutical agent design and therapy.

### SUMMARY OF THE INVENTION

Surprisingly it has now been found that single nucleotide polymorphisms in the NKNA gene can be used to determine the efficacy and compatibility of a pharmaceutically active compound, e.g. a NK-1 receptor antagonists, administered to a human being.

The present invention relates to a method for correlating single nucleotide polymorphisms in the NKNA gene with the efficacy and compatibility of a pharmaceutically active compound administered to a human being. The invention further relates to a method for determining the efficacy and compatibility of a pharmaceutically active compound administered to a human being which method comprises determining at least one single nucleotide polymorphism in the NKNA gene. Said methods are based on the determination of at least one single nucleotide polymorphism in the NKNA gene in the sample of said human being, which method comprises determining the nucleotide at position 41172 in intron 1 of the NKNA gene as defined by the position in FIG. 2 and determining the status of the human being by reference to polymorphism in the NKNA gene. Alternatively or, in addition thereto, the method comprises determining the sequence of the nucleic acid of the human being at positions 41112 in intron 1 of the NKNA gene, 37434 in intron 5 of the NKNA gene, 37114, 37025, 33949 in intron 6 of the NKNA gene and 33612 in the 3′UTR of the NKNA gene as defined by FIG. 2. The invention further relates to isolated nucleic acids comprising within their sequence the polymorphisms at the positions as defined before, to nucleic acid primers and oligonucleotide probes capable of hybridizing to such nucleic acids and to diagnostic kits comprising one or more of such primers and probes for detecting a polymorphism in the NKNA gene, to a pharmaceutical pack comprising NK-1 receptor antagonists and instructions for administration of the drug to human beings tested for the polymorphisms as well as to a computer readable medium with the stored sequence information for the polymorphisms in the NKNA gene.

The present invention is based on the discovery of single nucleotide polymorphisms in the NKNA gene.

The term “polymorphisms” is broadly defined to include all variations that are known to occur in nucleic acid sequences including insertions, deletions, substitutions and repetitive sequences including duplications.

“Polynucleotide” and “nucleic acid” refer to single or double-stranded molecules which may be DNA, comprised of the nucleotide bases A, T, C and G, or RNA, comprised of the bases A, U (substitutes for T), C, and G. The polynucleotide may represent a coding strand or its complement. Polynucleotide molecules maybe identical in sequence to the sequence which is naturally occurring or may include alternative codons which encode the same amino acid as that which is found in the naturally occurring sequence (See, Lewin “Genes V” Oxford University Press Chapter 7, 1994, 171-174. Furthermore, polynucleotide molecules may include codons which represent conservative substitutions of amino acids as described. The polynucleotide may represent genomic DNA or cDNA.

As defined herein, the “NKNA gene” is the sequence present within the nucleic acid sequences shown in FIG. 2 and in SEQ ID NO.1 located on human chromosome 7q21.1-q31.1. The NKNA gene includes 7 exon regions, 6 intron sequences intervening the exon sequences and 3′ and 5′ untranslated regions (3′UTR and 5′UTR) including the promotor element of the NKNA gene illustrated in FIG. 1. The first in frame ATG occurs in exon 2 (or at position 41031 in FIG. 2) while the TAG stop codon occurs in exon 7 (or at position 33724 in FIG. 2) for the putative 129 amino acid protein.

The present invention relates to a method for correlating single nucleotide polymorphisms in the NKNA gene with the efficacy and compatibility of a pharmaceutically active compound administered to a human being which method comprises determining single nucleotide polymorphisms in the NKNA gene of a human being and determining the status of said human being to which a pharmaceutically active compound was administered by reference to polymorphism in the NKNA gene.

According to a further aspect of the present invention there is provided a method for correlating single nucleotide polymorphisms in the NKNA gene with the efficacy and compatibility of a pharmaceutically active compound administered to a human being which method comprises determining single nucleotide polymorphisms in the NKNA gene of a human being and determining the status of said human being to which a pharmaceutically active compound was administered by reference to polymorphism at at least one or more positions in FIG. 2 comprising the NKNA gene including positions 33612, 33949, 37025, 37114, 37434, 41112 and/or 41172.

The status of the human being may be determined by reference to allelic variation at one, two, three, four, five, six or all seven positions. The status of the human being may also be determined by one or more of the specific polymorphisms identified herein in combination with one or more other single nucleotide polymorphisms.

The status of the human being comprises any response of the human being to the drug therapy, comprising physiological and psycological responses.

The term human being includes a human having or suspected of having a NK-1 receptor ligand mediated disease. At each position the human being may be homozygous for an allele or the human being may be heterozygous for an allele.

The allelic variation may have a direct effect on the response of an individual to drug therapy. The methods of the invention may therefore be useful both to predict the clinical response to such agents and to determine therapeutic dose.

Pharmaceutically active compounds may belong to the group of NK-1 receptor antagonists. Neurokinin receptor antagonists have been reviewed in Exp. Opin. Ther. Patents, 1996, 6, 367-378, and in Exp. Opin. Ther. Patents, 1997, 7, 43-54. The term “NK-1 receptor antagonist” as used herein refers to any natural or synthetic chemical compound that inhibits binding of substance P to the NK-1 receptor. A large number of such receptor antagonists are known and have been described. NK-1 receptor antagonists may be selected from the group consisting of 4-phenyl-pyridine derivatives, 3-phenyl-pyridine derivatives, 2-phenyl-substituted benzene derivatives, biphenyl derivatives, 4-phenyl-pyrimidine derivatives, 5-phenyl-pyrimidine derivatives, 1,4-diazepan-2,5-dione derivatives, 1,3,8-triaza-spiro[4.5]decan-4-one derivatives and piperidine derivatives as described in EP1035115, WO0050401, WO0050398, WO0053572, WO0073279, WO0073278, EP1103546, EP1103545, and WO0206236. These document as well as all documents referred to below are herewith incorporated by reference in their entirety.

Further preferred NK-1 receptor antagonists useful in connection with the present invention are the following NK-1 receptor antagonists currently under drug development:

GR205171: 3-Piperidinamine, N-[[2-methoxy-5-[5-(trifluoromethyl)-1H-tetrazol-1-yl]phenyl]methyl]-2-phenyl-, (2S-cis)- (Gardner et al. Regul. Pep. 65:45, 1996)

HSP-117: 3-Piperidinamine, N-[[2,3-dihydro-5-(1-methylethyl)-7-benzofuranyl]methyl]-2-phenyl-, dihydrochloride, (2S-cis)-

L 703,606: 1-Azabicyclo[2.2.2]octan-3-amine, 2-(diphenylmethyl)-N-[(2-iodophenyl)methyl]-, (2S-cis)-, oxalate (Cascieri et al., Mol. Pharmacol. 42, 458, 1992)

L 668,169: L-Phenylalanine, N-[2-[3-[[N-[2-(3-amino-2-oxo-1-pyrrolidinyl)-4-methyl-1-oxopentyl]-L-methionyl-L-glutaminyl-D-tryptophyl-N-methyl-L-phenylalanyl]amino]-2-oxo-1-pyrrolidinyl]-4-methyl-1-oxopentyl]-L-methionyl-L-glutaminyl-D-tryptophyl-N-methyl-, cyclic (8->1)-peptide, [3R-[1[S*[R* (S*)]],3R*]]-

LY 303241: 1-Piperazineacetamide, N-[2-[acetyl[(2-methoxyphenyl)methyl]amino]-1-(1H-indol-3-yl-methyl)ethyl]-4-phenyl-, (R)-

LY 306740: 1-Piperazineacetamide, N-[2-[acetyl[(2-methoxyphenyl)methyl]amino]-1-(1H-indol-3-yl-methyl)ethyl]-4-cyclohexyl-, (R)-

MK-869: 3H-1,2,4-Triazol-3-one, 5-[[2-[1-[3,5-bis(trifluoromethyl)phenyl]ethoxy]-3-(4-fluorophenyl)-4-morpholinyl]methyl]-1,2-dihydro-, [2R-[2α(R*),3α]]-

R-544: Ac-Thr-D-Trp(FOR)-Phe-N-MeBzl

Spantide III: L-Norleucinamide, N6-(3-pyridinylcarbonyl)-D-lysyl-L-prolyl-3-(3-pyridinyl)-L-alanyl-L-prolyl-3,4-dichloro-D-phenylalanyl-L-asparaginyl-D-tryptophyl-L-phenylalanyl-3-(3-pyridinyl)-D-alanyl-L-leucyl-

WIN-62,577: 1H-Benzimidazo[2,1-b]cyclopenta[5,6]naphtho[1,2-g]quinazolin-1-ol, 1-ethynyl-2,3,3a,3b,4,5,15,15a,15b,16,17,17a-dodecahydro-15a,17a-dimethyl-, (1R,3aS,3bR,15aR,15bS,17aS)-GR 103,537

L 758,298: Phosphonic acid, [3-[[2-[1-[3,5-bis(trifluoromethyl)phenyl]ethoxy]-3-(4-fluorophenyl)-4-morpholinyl]methyl]-2,5-dihydro-5-oxo-1H-1,2,4-triazol-1-yl]-, [2R-[2α(R*),3α]]-

NKP608: (2R,4S)-N-[1-{3,5-bis(trifluormethyl)-benzoyl}-2-(4-chloro-benzyl)-4-piperidinyl]-quinoline-4-carboxamide

CGP49823: (2R, 4S)-2-benzyl-1-(3,5-dimethylbenzoyl)-N-[(4-quinolinyl)methyl]-4-piperineamine) dihydrochloride

CP-96,345: 2S, 3S)-cis-(2(diphenylmethyl)-N-[(2-methoxyphenyl)methyl]-1-azabicyclo[2.2.2]octan-3-amine (Srider et al., Science 251:435, 1991)

CP-99,994: ((2S, 3S)-cis-3-(2-methoxybenzylamino)-2-phenyl-piperidine)dihydrochloride (Desai et al., J. Med. Chem. 35:4911, 1992)

CP-122,721: (+)-2S, 3S)-3-(2-methoxy-5-trifluoromethoxybenzyl)amino-2-phenylpiperidine

FK 888: (N2-[(4R)-4-hydroxy-1(1-methyl-1H-indol-3-yl)cabonyl-L-propyl\-N-methyl-N-phenylmethyl-L-3-(2-naphthyl)-alaninamide (Fujii et al., Br. J. Pharm. 107:785, 1992)

GR203040: (2S, 3S and 2R, 3R)-2methoxy-5-tetrazol-1-yl-benzyl-(2-phenyl-piperidin-3-yl)-amine

GR 82334: [D-Pro9,] spiro-gamma-lactam]Leu10, Trp11]physalaemin-(1-11)

GR 94800: PhCO-Ala-Ala-DTrp-Phe-DPe-DPro-Pro-NIe-NH2

L 732,138: N-acetyl-L-tryptophan

L 733,060: ((2S,S)-3-((3,5-bis(trifluoromethyl)phenyl)methyloxy)-2-phenyl piperidine

L 742,694: (2-(S)-(3.5-bis(trifluromethyl)benzyloxy)-3-(S)-phenyl-4-(5-(3-oxo-1,2,4-triazolo)methylmorpholine

L 754,030: 2-(R)-(1-(R)-3,5-bis(trifluoromethyl)phenylethoxy)-3-(S)-(4-fluoro)phenyl-4-(3-oxo-1,2,4-triazol-5-yl)methylmorpholine

LY 303870: (R)-1[N-(2-methoxybenzyl)acetylamino]-3-(1H-indol-3-yl)-2-[N-(2-(4-(pi peridinyl)piperidin-1-yl)acetyl)amino]propane

MEN 11149: 2-(2-naphthyl)-1-N-[(1R, 2S)-2-N-[2(H)indol-3-ylcarbonyl]aminocyclohexanecarbonyl]-1-[N′-ethyl-N′-(4-methylphenylacetyl)]diaminoethane (Cirillo et al., Eur. J. Pharm. 341:201, 1998)

PD 154075: (2-benzofuran)-CH2OCO]-(R)-alpha-MeTrp-(S)-NHCH(CH3)Ph

RP-67580: (3aR, 7aR)-7,7-diphenyl-2[1-imino-2(2-methoxyphenyl)-ethyl]+++perhydroisoidol-4-one hydrochloride (Garret et al., PNAS 88:10208, 1991)

RPR 100893: (3aS, 4S, 7aS)-7,7-diphenyl-4-(2-methoxyphenyl)-2-[(S)-2-(2-methoxyphenyl)proprionyl]perhydroisoindol-4-ol

Spendide: Tyr-D-Phe-Phe-D-His-Leu-Met-NH2

Spantide II: D-NicLys1, 3-PaI3, D-CI2Phe5, Asn6, D-Trp7.0, NIe11-substance P

SR140333: (S)-1-[2-[3-(3, 4-dichlorphenyl)-1(3-isopropoxyphenylacetyl) piperidin-3-yl]ethyl]-4-phenyl-1 azaniabicyclo [2.2.2]octane (Edmonts et al., Eur. J. Pharm. 250:403, 1993)

WIN-41,708: (17beta-hadroxy-17alpha-ethynyl-5alpha-androstano[3.2-b]pyrimido[1,2-a]benzimidazole

WIN-62,577: 1H-Benzimidazo[2,1-b]cyclopenta[5,6]naphtho[1,2-g]quinazolin-1-ol, 1-ethynyl-2,3,3a,3b,4,5,15,15a,15b,16,17,17a-dodeachydro-15a,17a-dimethyl-,(1R, 3aS, 3bR,15aR, 15bS, 17aS)-

SR-48,968: (S)-N-methyl-N [4-(4-acetylamino-4-[phenylpiperidino)-2-(3,4-dichlorophenyl)-butyl]benzamide

L-659,877: cyclo[Gln, Trp, Phe, Gly, Leu, Met]

MEN 10627: cyclo(Met-Asp-Trp-Phe-Dap-Leu)cyclo(2beta-5beta)

SR 144190: (R)-3(1-[2-(4-benzoyl-2-(3,4-difluorophenyl)-morpholin-2-yl)ethyl]-4-phenylpiperidin-4-yl)-1-dimethylurea

GR 94800: PhCO-Ala-Ala-D-Trp-Phe-D-Pro-Pro-NIe-NH2

SR-142,801: (S)-(N)-1-(3-(1-benzoyl-3-(3,4-dichlorophenyl)piperidin-3-yl)propyl)-4-phenylpiperidin-4-yl)-N-methyl acetamide

R820: 3-indolylcarbonyl-Hyp-Phg-N(Me)-Bzl

R486: H-Asp-Ser-Phe-Trp-beta-Ala-Leu-Met-NH2

SB 222200: (S)-(−)-N-(a-ethylbenzyl)-3-methyl-2-phenylquinoline-4-carboximide

L 758,298: Phosphonic acid, [3-[[2-[1-[3,5-bis(trfluoromethyl)phenyl]ethoxy]-3-(4-fluorophenyl)-4-morpholinyl]-2, 5-dihydro-5oxo-1H-1,2,4-triazol-1-yl]-, [2R-[2a(R*), 3α]]-

NK-608: (2R,4S)-N-[1-{3,5-bis(trifluormethyl)-benzoyl}-2-(4-chloro-benzyl)-4-piperidinyl]-quinoline-4-carboxamide

CGP 47899: Shilling et al., Pers. Med. Chem. 207, 1993

MEN 11467: Evangelista et al., XXIX Nat. Congr. of the Ital. Pharmacological Soc., Florence 20-23.06.1999.

Any reference herein to the compound specifically named above includes also the pharmaceutically acceptable acid addition salts thereof.

Further information on these NK-1 receptor antagonists under drug development can be found in the published literature.

Additional suitable NK-1 receptor antagonists are described in the following published patents and patent applications.

U.S. Pat. No. 5,990,125 in particular the compounds Ia to Ie, X and XVI to XXI, as well as other antagonists comprising quinuclidine, piperidine ethylene diamine, pyrrolidine and azabornane derivatives and related compounds that exhibit activity as substance P receptor antagonists as described in column 33 of U.S. Pat. No. 5,990,125. These antagonists are preferably used in dosages as specified in column 34 of U.S. Pat. No. 5,990,125.

Further suitable NK-1 receptor antagonists are described in the following publications:

U.S. Pat. Nos. (USP)

| | | | |
|---|---|---|---|
| 5,977,104 | 5,162,339 | 4,481,139 | 5,232,929 |
| 5,998,444 | 5,242,930 | 5,373,003 | 5,981,744 |
| 5,387,595 | 5,459,270 | 5,494,926 | 5,496,833 |
| 5,637,699 | | | |

Europ. Patent Application, Publ. Nos. (EP-A-)

| | | | |
|---|---|---|---|
| 0 360 390 | 0 394 989 | 0 428 434 | 0 429 366 |
| 0 430 771 | 0 436 334 | 0 433 132 | 0 482 539 |
| 0 498 069 | 0 499 313 | 0 512 901 | 0 512 902 |
| 0 514 273 | 0 514 274 | 0 514 275 | 0 514 276 |
| 0 515 681 | 0 517 589 | 0 520 555 | 0 522 808 |
| 0 528 495 | 0 532 456 | 0 533 280 | 0 536 817 |
| 0 545 478 | 0 558 156 | 0 577 394 | 0 585 913 |
| 0 590 152 | 0 599 538 | 0 610 793 | 0 634 402 |
| 0 686 629 | 0 639 489 | 0 694 535 | 0 699 655 |
| 0 699 674 | 0 707 006 | 0 708 101 | 0 709 375 |
| 0 709 376 | 0 714 891 | 0 723 959 | 0 733 632 |
| 0 776 893 | | | |

PCT Int. Patent Publ. Nos.(WO)

| | | | |
|---|---|---|---|
| 90/05525 | 90/05729 | 91/09844 | 91/18899 |
| 92/01688 | 92/06079 | 92 12151 | 92/15585 |
| 92/17449 | 92/20661 | 92/20676 | 92/21677 |
| 92/22569 | 93/00330 | 93/00331 | 93/01159 |
| 93/01165 | 93/01169 | 93/01170 | 93/06099 |
| 93/09116 | 93/10073 | 93/14084 | 93/14113 |
| 93/18023 | 93/19064 | 93/21155 | 93/21181 |
| 93/23380 | 93/24465 | 94/00440 | 94/01402 |
| 94/02461 | 94/02595 | 94/03429 | 94/03445 |
| 94/04494 | 94/04496 | 94/05625 | 94/07843 |
| 94/08997 | 94/10165 | 94/10167 | 94/10168 |
| 94/10170 | 94/11368 | 94/13639 | 94/13663 |
| 94/14767 | 94/15903 | 94/19320 | 94/19323 |
| 94/20500 | 94/26735 | 94/26740 | 94/29309 |
| 95/02595 | 95/04040 | 95/04042 | 95/06645 |
| 95/07886 | 95/08908 | 95/08549 | 95/11880 |
| 95/14017 | 95/15311 | 95/16679 | 95/17382 |
| 95/18124 | 95/18129 | 95/19344 | 95/20575 |
| 95/21819 | 95/22525 | 95/23798 | 95/26338 |
| 95/28418 | 95/30674 | 95/30687 | 95/33744 |
| 96/05181 | 96/05193 | 96/05203 | 96/06094 |
| 96/07649 | 96/10562 | 96/16939 | 96/18643 |
| 96/20197 | 96/21661 | 69/29304 | 96/29317 |
| 96/29326 | 96/29328 | 96/31214 | 96/32385 |
| 96/37489 | 97/01553 | 97/01554 | 97/03066 |
| 97/08144 | 97/14671 | 97/17362 | 97/18206 |
| 97/19084 | 97/19942 | 97/21702 | 97/49710 |

British Patent Publ. Nos. (GB)

| | | | |
|---|---|---|---|
| 2 266 529 | 2 268 931 | 2 269 170 | 2 269 590 |
| 2 271 774 | 2 292 144 | 2 293 168 | 2 293 169 |
| 2 302 689 | | | |

Indications for the NK-1 receptor antagonists described above are treatment of pain, headache, especially migraine, Alzheimer's disease, disorders of the central nervous system such as certain depressive disorders, anxiety, and emesis, psychosis, multiple sclerosis, attenuation of morphine withdrawal, cardiovascular changes, oedema, such as oedema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases, benign prostatic hyperplasia, motion sickness, treatment induced vomiting, cancer such as malignant gliomas, traumatic brain injury.

Preferably, the NK-1 receptor antagonist is 2-(3,5-bis-trifluoromethyl-phenyl)-N-[6-(1,1-dioxo-1λ⁶-thiomorpholin-4-yl)-4-o-tolyl-pyridin-3-yl]-N-methyl-isobutyramide or 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide as disclosed in EP1035115.

Most preferably, the NK-1 receptor antagonist is 2-(3,5-bis-trifluoromethyl-phenyl)-N-methyl-N-(6-morpholin-4-yl-4-o-tolyl-pyridin-3-yl)-isobutyramide as disclosed in EP1035115.

Preferred indications for these NK-1 receptor antagonists are those, which include disorders of the central nervous system, for example the treatment or prevention of certain depressive disorders, anxiety or emesis. A major depressive episode has been defined as being a period of at least two weeks during which, for most of the day and nearly every day, there is either depressed mood or the loss of interest or pleasure in all, or nearly all activities.

In another aspect the present invention provides a method for determining the efficacy and compatibility of a pharmaceutically active compound for a human being, which method comprises determining the presence of single nucleotide polymorphisms in the NKNA genomic sequence obtained from the human being which single nucleotide polymorphisms are correlated with the efficacy and compatibility of the pharmaceutically active compound, and thereby determining the efficacy and compatibility of the pharmaceutically active compound for the human being.

Preferably, the present invention relates to a method for determining the efficacy and compatibility of a pharmaceutically active compound for a human being, which method comprises determining the nucleotide at at least one or more of the positions 33612, 33949, 37025, 37114, 37434, 41112 and/or 41172 of the nucleotide sequence in FIG. 2 comprising the NKNA gene in the NKNA genomic sequence obtained from the human being which single nucleotide polymorphisms are correlated with the efficacy and compatibility of the pharmaceutically active compound, and thereby determining the efficacy and compatibility of the pharmaceutically active compound for the human being.

Preferably, the present invention relates to a method for determining the efficacy and compatibility of a pharmaceutically active compound for a human being, which method comprises determining in the NKNA genomic sequence obtained from the human being the presence of a single nucleotide polymorphism selected from the group of a C or a T at position 33612 of the nucleotide sequence in FIG. 2 comprising the NKNA gene, a T or a C at position 33949 in FIG. 2, a C or a T at position 37025 in FIG. 2, a G or an A at position 37114 in FIG. 2, an A or a C at position 37434 in FIG. 2, a T or a G at position 41112 in FIG. 2, a G or an A at position 41172 in FIG. 2, and combinations thereof as well as their reverse complement, which single nucleotide polymorphisms are correlated with the efficacy and compatibility of the pharmaceutically active compound, and thereby determining the efficacy and compatibility of the pharmaceutically active compound for the human being.

In this method, the pharmaceutically active compound may be a NK-1 receptor antagonist. The NK-1 receptor antagonist may be any NK-1 receptor antagonist as described beforehand.

The method in accordance with the present invention can be performed using any suitable method for detecting single nucleotide variations, such as e.g. direct mass-analysis of PCR products using mass spectrometry, direct analysis of invasive cleavage products, direct sequence analysis, allele specific amplification (i.e. ARMS™-allele specific amplification; ARMS referring to amplification refractory mutation system), ALEX™ (amplification refractory mutation system linear extension) and COPS (competitive oligonucleotide priming system), allele specific hybridization (ASH), oligonucleotide ligation assay (OLA), Invader Assay, DNA chip analysis and restriction fragment length polymorphism (RFLP) (for review see Genome Research, 1998, 8, 769-776; Pharmacogenomics, 2000, 1, 95-100; Human Mutation, 2001, 17, 475-492).

The test sample of the nucleic acid carrying the said polymorphism is conveniently a sample of blood, bronchoalveolar lavage fluid, sputum, urine or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique, e.g. polymerase chain reaction (PCR) or ligase chain reaction (LCR), before analysis of allelic variation.

Polymorphisms in the NKNA gene can be identified by sequencing a nucleic acid sample of patients and comparing the sequence to controls or by PCR-amplification of 400-600 base pair fragments (covering coding and regulatory regions of the NKNA gene) in the DNA of unrelated individuals of different ethnic origin. Fragments can be sequenced in these samples with a forward and reverse primer, polymorphisms can be detected by using the PolyPhred software (licensed from University of Washington) and allele frequencies for the variants can be established (Human Mutation, 2001, 17,243-254).

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. International patent application WO 00/06768 lists a number of amplification techniques and mutation detection techniques, some based on PCR. These may be used in combination with a number of signal generation systems, a selection of which is also listed in WO 00/06768. Many current methods for the detection of allelic variation are reviewed by Nollau et al., Clin. Chem., 1997, 43, 1114-1120; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

The invention further provides an isolated nucleic acid molecule selected from the following polymorphism containing sequences:

the nucleic acid sequence of FIG. 2 with T at position 33612 as defined by the position in FIG. 2;

the nucleic acid sequence of FIG. 2 with C at position 33949 as defined by the position in FIG. 2;

the nucleic acid sequence of FIG. 2 with T at position 37025 as defined by the position in FIG. 2;

the nucleic acid sequence of FIG. 2 with A at position 37114 as defined by the position in FIG. 2;

the nucleic acid sequence of FIG. 2 with C at position 37434 as defined by the position in FIG. 2;

the nucleic acid sequence of FIG. 2 with G at position 41112 as defined by the position in FIG. 2;

the nucleic acid sequence of FIG. 2 with G at position 41172 as defined by the position in FIG. 2; or

a complementary strand thereof or a fragment thereof of at least 20 bases comprising at least one of the polymorphisms.

An “isolated” NKNA nucleic acid molecule is a nucleic acid molecule that is identified and separated from at least one contaminant nucleic acid molecule with which it is ordinarily associated in the natural source of the NKNA nucleic acid. An isolated NKNA nucleic acid molecule is other than in the form or setting in which it is found in nature. Isolated NKNA nucleic acid molecules therefore are distinguished from the NKNA nucleic acid molecule as it exists in natural cells. However, an isolated NKNA nucleic acid molecule includes NKNA nucleic acid molecules contained in cells that ordinarily express NKNA where, for example, the nucleic acid molecule is in a chromosomal location different from that of natural cells.

Furthermore the invention relates to allele-specific nucleic acid primers which can be used as diagnostic primers for detecting a polymorphism in the NKNA gene capable of hybridizing to nucleic acids comprising within their sequence the polymorphisms at positions 33612 in FIG. 2, 33949 in FIG. 2, 37025 in FIG. 2, 37114 in FIG. 2, 37434 in FIG. 2, 41112 in FIG. 2, and 41172 in FIG. 2.

Another aspect of the present invention is a nucleic acid primer comprising the following sequences selected from the group of:

the nucleic acid sequence as defined by SEQ ID NO.8;

the nucleic acid sequence as defined by SEQ ID NO.9;

the nucleic acid sequence as defined by SEQ ID NO.10;

the nucleic acid sequence as defined by SEQ ID NO.11;

the nucleic acid sequence as defined by SEQ ID NO.12;

the nucleic acid sequence as defined by SEQ ID NO.13;

the nucleic acid sequence as defined by SEQ ID NO.14;

the nucleic acid sequence as defined by SEQ ID NO.15; or

the nucleic acid sequence as defined by SEQ ID NO.16 and their reverse complement.

An allele specific primer is used, generally together with a constant primer, in an amplification reaction such as a PCR reaction, which provides the discrimination between alleles through selective amplification of one allele at a particular sequence position e.g. as used for ARMS™ assays. The length of the allele specific primer is preferably 17-50 nucleotides, more preferably about 17-35 nucleotides, most preferably about 17-30 nucleotides.

Preferably, the allele specific primer corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer. Often the nucleotide at the −2 and/or −3 position (relative to the 3′ terminus) is mismatched in order to optimize differential primer binding and preferential extension from the correct allele discriminatory primer only.

The invention also relates to oligonucleotide probes for detecting a polymorphism in the NKNA gene capable of hybridizing specifically to a nucleic acid comprising within its sequence the polymorphism at positions 33612 in FIG. 2, 33949 in FIG. 2, 37025 in FIG. 2, 37114 in FIG. 2, 37434 in FIG. 2, 41112 in FIG. 2, and 41172 in FIG. 2.

The term “oligonucleotide probe” refers to a nucleotide sequence of at least 17 nucleotides in length which corresponds to part or all of the human NKNA gene, preferably a part of the human NKNA gene which expresses the polymorphism. A length of 17 to 50 nucleotides is preferred. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection, such as in Molecular Beacons.

The present invention also encompasses a diagnostic kit comprising one or more nucleic acid primer(s) and/or one or more oligonucleotide probe(s) with a single nucleotide polymorphism of the NKNA gene selected from the group consisting of a C or a T at position 33612 in FIG. 2, a T or a C at position 33949 in FIG. 2, a C or a T at position 37025 in FIG. 2, a G or an A at position 37114 in FIG. 2, an A or a C at position 37434 in FIG. 2, a T or a G at position 41112 in FIG. 2, and a G or an A at position 41172 in FIG. 2, and combinations thereof as well as their reverse complement.

The present invention further provides a pharmaceutical pack comprising NK-1 receptor antagonists and instructions for administration of the drug to human beings tested for a single nucleotide polymorphism at one or more positions of the NKNA gene.

The present invention further provides a pharmaceutical pack comprising NK-1 receptor antagonists, preferably 2-(3,5-bis-trifluoromethyl-phenyl)-N-methyl-N-(6-morpholin-4-yl-4-o-tolyl-pyridin-3-yl)-isobutyramide, and instructions for administration of the drug to human beings tested for a single nucleotide polymorphism at one or more positions of the NKNA gene according to a method of the present invention.

The present invention further provides the use of a NK-1 receptor antagonist for the preparation of a medicament for treating a NK-1 receptor ligand-mediated disease in a human being diagnosed as having a single polynucleotide polymorphism at one or more of position 33612, 33949, 37025, 37114, 37434, 41112 and 41172 in FIG. 2 comprising the NKNA gene.

The present invention also includes a computer readable medium having stored thereon sequence information for the polymorphisms in the NKNA gene including polymorphisms at positions 33612 in FIG. 2, 33949 in FIG. 2, 37025 in FIG. 2, 37114 in FIG. 2, 37434 in FIG. 2, 41112 in FIG. 2, and 41172 in FIG. 2.

A method for performing sequence identification is also provided in the present invention, said method comprising the steps of providing a nucleic acid sequence selected from the group consisting of the nucleic acid sequence of FIG. 2 with T at position 33612; the nucleic acid sequence of FIG. 2 with C at position 33949; the nucleic acid sequence of FIG. 2 with T at position 37025; the nucleic acid sequence of FIG. 2 with A at position 37114; the nucleic acid sequence of FIG. 2 with C at position 37434; the nucleic acid sequence of FIG. 2 with G at position 41112; the nucleic acid sequence of FIG. 2 with G at position 41172; or a complementary strand thereof or a fragment thereof of at least 20 bases comprising at least one of the polymorphisms, and comparing said nucleic acid sequence to at least one other nucleic acid or polypeptide sequence to determine identity.

Having now generally described this invention, the same will become better understood by reference to the specific examples, which are included herein for purpose of illustration only and are not intended to be limiting unless otherwise specified, in connection with the following figures:

### BRIEF DESCRIPTION OF THE DRAWINGS

FIG. 1: Genomic structure of the NKNA gene and polymorphisms found in the gene. Exon-intron boundaries are indicated with respect to the sequence depicted in FIG. 2 (corresponding to parts of the DNA sequence of the accession no. EM_HUM 1:AC004140.1). Positions of polymorphisms are indicated with arrows.

FIG. 2: Part of the genomic sequence of the PAC clone DJ0841B21 defined by the accession no. EM_HUM1:AC004140.1 containing the preprotachykinin gene. Exon-intron boundaries are indicated in FIG. 1. The sequence corresponds to SEQ ID NO.1 whereas position 33301 corresponds to position 1 in SEQ ID NO.1, and position 41800 corresponds to position 8500 in SEQ ID NO.1.

FIG. 3: Identified single nucleotide polymorphisms in the NKNA gene with positions as defined in FIG. 2.

FIG. 4: 2×2 Contingency table for 29 position 41172 genotype test results with concentration before emesis test >20 ng/ml 2-(3,5-bis-trifluoromethyl-phenyl)-N-methyl-N-(6-morpholin-4-yl-4-o-tolyl-pyridin-3-yl)-isobutyramide. Effect=yes: number of vomits+retches≦3. Fisher's Exact Test (two-sided): p=0.033. The genotypes of the subjects were classified according to the following categories: 0-2 are allele 2 (nucleotide G) homozygotes (two copies of allele 2, no copies of allele 1); not 0-2 include allele 1 (nucleotide A) homozygotes 2-0 (two copies of allele 1, no copies of allele 2) and heterozygotes 1-1 (one copy of allele 1 and one copy of allele 2).

### DETAILED DESCRIPTION OF THE INVENTION

### EXAMPLES

Commercially available reagents referred to in the Examples were used according to manufacturer's instructions unless otherwise indicated.

### Example 1

### Detection of Polymorphisms

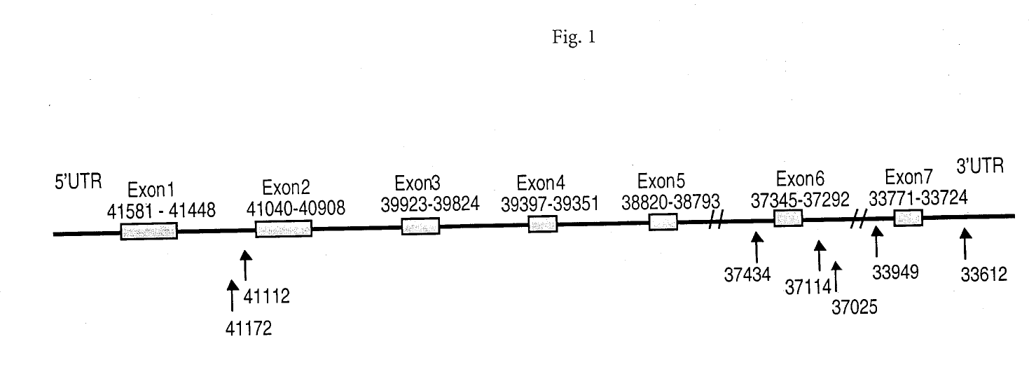

For all single nucleotide polymorphisms discovery was performed by double-stranded DNA sequencing using an ABI capillary sequencer and Big Dye chemistry (ABI). First the genomic organization of the NKNA gene was derived from a PAC clone found in the EMBL database with the accession no. EM_HUM1:AC004140.1 by a BLAST search with the NKNA mRNA (accession no. U37529.1 in the EMBL database). Exon-intron boundaries were derived as indicated in FIG. 1 and primers were designed to amplify all coding and regulatory regions of the gene. The primers used to amplify all exons are shown below and were also used as sequencing primers. All polymorphisms were targeted with these pair-of-primer sets:

TABLE 1
List of oligonucleotide primers
for polymorphism detection

| Primer type | Nucleotide sequence | SEQ ID NO |
|---|---|---|
| Primer 1 | CATGTTTACAATACATATTGGCAC | SEQ ID NO. 2 |
| Primer 2 | GTATATGATGAATGATG | SEQ ID NO. 3 |
| Primer 3 | CACCCTCATTCTTCCCTGC | SEQ ID NO. 4 |
| Primer 4 | CTTCAGTCTCACCAAAACTTG | SEQ ID NO. 5 |
| Primer 5 | GTGCCCCTTTCCATCCTCTC | SEQ ID NO. 6 |
| Primer 6 | GGTGTGGGTTGGTGGGTTAG | SEQ ID NO. 7 |

To detect polymorphisms the NKNA gene was PCR-amplified from 47 unrelated individuals of 5 different ethnic origins. Using fragment-specific primer pairs (length 18-27 bp), 200-700 bp fragments were amplified e.g. a 519 bp-PCR product was generated with the primer pair 5 and 6. Fragments were designed covering coding and regulatory regions of the NKNA gene. After a column purification of the PCR products, the DNA was sequenced on an ABI capillary sequencer using ABI Dye terminator chemistry (fluorescence based sequencing). Polymorphisms in the DNA sequences were detected using Polyphred software (Nickerson, D. et al. 1997: NAR 25(14): 2745-2751), which operates on the basis of Phred, Phrap and Consed (programs all licensed from the University of Washington, USA). This program is able to automatically detect the presence of heterozygous single nucleotide substitutions by fluorescence-based sequencing. In the example above the following 2 polymorphisms were detected in the 519 bp fragment:

| | |
|---|---|
| 41112* | T or G |
| 41172* | G or A |

*defined by the position in accession no. EM_HUM1:AC004140.1

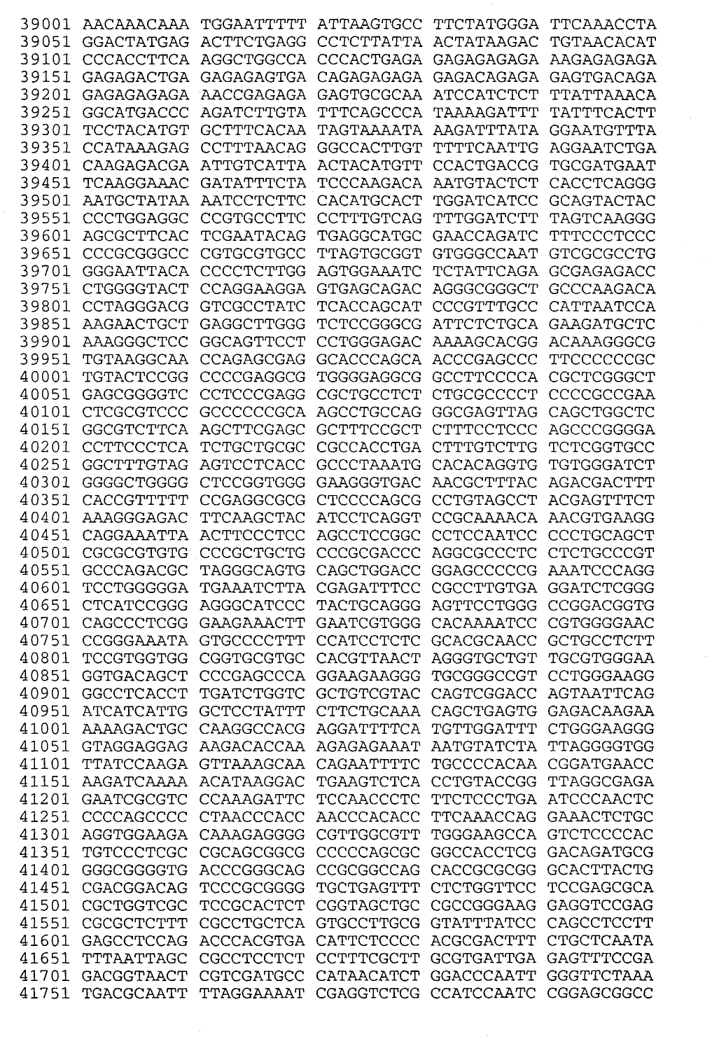

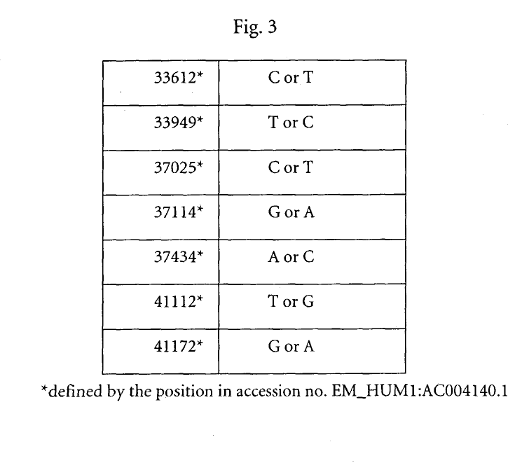

In total, seven single polynucleotide polymorphisms were detected in the NKNA gene as shown in FIG. 3.

### Example 2

### Genotyping

Selection of Subjects

The study protocol and the informed consent form were submitted for approval to the local ethical committee. All subjects provided written informed consent for their blood sample to be used for genotyping. The consent could be withdrawn up to a month later, if the subjects changed their mind.

All the samples were assigned new independent codes and within six months after clinical database closure the link between the new and original codes was deleted. This was an added measure to ensure patient confidentiality; however, as a consequence it is not possible to retrieve genotype information based on the patient's name or number used in the original clinical trial. In approximately 15 years time, all blood and DNA samples will be destroyed.

Genotyping Assay

Single blood samples (9 ml) were collected in EDTA tubes. These were frozen and stored between −20 and −70 C, before being sent to the Roche Central Sample Office (CSO) in Basel, Switzerland, where they were aliquoted into three tubes and assigned new, independent codes on bar code labels to assure patient anonymity. Two samples of blood (1 ml and 4 mls) were sent to the Roche Sample Repository (RSR) at Roche Molecular Systems (RMS) in Alameda, Calif., and stored at −80° C. The remaining 4 ml aliquot was stored at −80° C. in the CSO in Basel, Switzerland. All procedures performed on the samples at the RSR were done according to established standard operating procedures in compliance with GCP guidelines.

DNA was extracted from 400 μl of the whole blood using a silica membrane-based extraction method (QiaAmp 96 DNA Blood kit, Valencia, Calif.). Controls included 10 mM Tris pH 8.0, 0.1 mM EDTA (TE) buffer and whole blood from a blood unit with a known yield of DNA.

Three genetic markers were selected based on the results from polymorphism discovery in the NKNA gene. Samples were genotyped for these single nucleotide polymorphisms by a kinetic PCR method described by Germer et al., Genome Res. (2000), 10, 258-266 with the modification of using single sample for each reaction instead of pooling samples. This method allows discrimination of single nucleotide polymorphisms without the use of fluorescent probes.

In the kinetic thermal cycler (KTC) format, the generation of double-stranded amplification product is monitored using a DNA intercalating dye and a thermal cycler which has a fluorescence-detecting CCD camera attached (PE-Biosystems GeneAmp 5700 Sequence Detection System). Fluorescence in each well of the PCR amplification plate is measured at each cycle of annealing and denaturation. The cycle at which the relative fluorescence reached a threshold of 0.5 using the SDS software from PE-Biosystems was defined as the Cₜ.

The amplification reactions were designed to be allele-specific, so that the amplification reaction was positive if the allele was present and the amplification reaction was negative if the allele was absent. For each bi-allelic polymorphism, one well of the amplification plate was set up to be specific for allele 1 and a second well was set up to be specific for allele 2. For each polymorphism to be detected, three primers were designed—two allele-specific primers and one common primer (Table 2). Reactions for allele 1 contained allele 1-specific primer and the common primer and reactions for allele 2 contained allele 2-specific primer and the common primer. The Cₜ values for each pair of wells is used to calculate the delta Cₜ which is used to determine the allele call.

TABLE 2
List of oligonucleotide primers used for polymorphism screening

| | | | | Primer | |
|---|---|---|---|---|---|
| | Primer | | SEQ ID | concentration | Annealing |
| Marker | ID | Nucleotide sequence | NO | (in μM) | temperature |
| 411172 | PPT1/ | CCGGTACAGGTGAGACTTT | SEQ ID | 0.4 | 58° C. |
| | 411172 | | NO.8 | | |
| 411172 | PTT2/ | CCGGTACAGGTGAGACTTC | SEQ ID | 0.4 | 58° C. |
| | 41172 | | NO.9 | | |
| 411172 | PTT3/ | CAACGGATGAACCAAGATC | SEQ ID | 0.4 | 58° C. |
| | 41172 | | NO.10 | | |
| 37114 | PTT4/ | AGAGAAATAGACAGATACTGTGGTAG | SEQ ID | 0.2 | 58° C. |
| | 37114 | | NO.11 | | |
| 37114 | PPT5/ | AGAGAAATAGACAGATACTGTGGTAA | SEQ ID | 0.2 | 58° C. |
| | 37114 | | NO.12 | | |
| 37114 | PTT6/ | ATGGATTTATAGCTGGTTAAGC | SEQ ID | 0.2 | 58° C. |
| | 37114 | | NO.13 | | |
| 37025 | PTT7/ | CAGATCTATAGGAAAGAATATAGCAC | SEQ ID | 0.2 | 58° C. |
| | 37025 | | NO.14 | | |
| 37025 | PTT8/ | CAGATCTATAGGAAAGAATATAGCAT | SEQ ID | 0.2 | 58° C. |
| | 37025 | | NO.15 | | |
| 37025 | PTT9/ | CCATTTAATCATTACCAACCTGAATC | SEQ ID | 0.2 | 58° C. |
| | 37025 | | NO.16 | | |

The amplification conditions were as follows: 10 mM Tris pH 8.0, 40 mM KCl, 2 mM MgCl₂, 50 μm each of dATP, dCTP, and dGTP, 25 μm of dTTP and 75 μm of dUTP, 4% DMSO, 0.2×SYBR Green I (Molecular Probes, Eugene, Oreg.), 2% glycerol, 2 units of uracil N-glycosylase (UNG), 15 units of Stoffel Gold DNA polymerase (for reference see Nature (1996), 381, 445-6) and primers in an 85 μl volume for each well. The concentration of the primers used for each assay are listed in Table 2. 30 ng of DNA in a 15 μl volume was then added to each well.

To reduce the possibility of contamination by pre-existing amplification product, the assay procedure included the incorporation of dUTP into the amplification product and an incubation step for UNG degradation of pre-existing dU-containing products (Longo et al, Gene (1990), 93,125-128).

Amplification reactions were prepared using an aliquoting robot (Packard Multiprobe II, Meriden, Conn.) in 96-well amplification plates identified by barcode labels generated by the experiment management database. Parameters for procedures performed by the robot were set to minimize the possibility of cross-contamination. For each plate of 81 samples, 5 samples were run in duplicate and the duplicate results were analyzed to determine that they matched.

The thermal cycling conditions were as follows: 2 minutes at 50° C. for UNG degradation of any previously contaminating PCR products, 12 minutes at 95° C. for Stoffel Gold polymerase activation, 55 cycles of denaturation at 95° C. for 20 seconds and annealing at 58° C. for 20 seconds, followed by a dissociation step of 0.57 minute at 1 degree increments from 60° C. to 95° C. The amplification reactions were run in PE Biosystems GeneAmp 5700 Sequence Detection Systems (SDS) instruments (Foster City, Calif.). The first derivatives of the dissociation curves were produced by the SDS software and examined as needed to confirm that the fluorescence in a given reaction was due to amplification of a specific product with a well-defined dissociation peak rather than non-specific primer-dimer. Product differentiation was done by Analysis of DNA Melting Curves during PCR following the method of K. M. Ririe et al., Anal. Biochem. (1997), 245, 154-160.

The Cₜ of each amplification reaction was determined and the difference between the Cₜ for allele 1 and allele 2 (delta Cₜ) was used as the assay result. Samples with delta Cₜs between −3.0 and 3.0 were considered heterozygous (A1/A2). Samples with delta Cₜs below −3.0 were considered homozygous for A1 (A1/A1); samples with delta Cₜs above 3.0 were considered homozygous for A2 (A2/A2). In most cases, the delta Cₜ differences between the three groups of genotypes were well-defined and samples with Cₜ values close to 3.0 were re-tested as discrepants.

Each assay was run on a panel of 20 cell line DNAs to identify cell lines with the appropriate genotypes for use as controls on each assay plate (A1/A1, A1/A2, and A2/A2). The cell line DNA was obtained from the Culture Collection in R & D Service, Roche Molecular Systems (RMS) Alameda, CA and was extracted using the Qiagen extraction kits (QiaAmp DNA Blood kits, Valencia, Calif.). The genotypes of the cell line DNAs were confirmed by DNA sequencing. Three cell line DNAs (A1/A1, A1/A2, and A2/A2) were run as controls on each plate of clinical trial samples and used to determine the between-plate variability. The Cₜ values obtained for the control cell lines were analyzed to determine the cutoff for the delta Cₜ values obtained for the clinical trial samples.

A data file containing the Cₜ values for each well was generated by the SDS software for each plate and entered into the experiment management database. For all the SNP assays ran for the clinical trial, a data file with Cₜ values for all the samples identified by the independent code was extracted from the database and interpreted to the final genotypes by a in-house developed program. The genotype results were sent to the statistician and matched to the clinical data also identified by the independent code for statistical analysis.

### Example 3

### Emesis Test

The described emesis test was performed in two studies. A Single Ascending Dose study (SAD) and a Multiple Ascending Dose study (MAD). In the SAD the emesis test was performed 6 and/or 24 hrs after intake of 2-(3,5-bis-trifluoromethyl-phenyl)-N-methyl-N-(6-morpholin-4-yl-4-o-tolyl-pyridin-3-yl)-isobutyramide. In the MAD the emesis test was performed after 14 once daily doses, 6 or 24 hrs after the last dose. SAD

In the SAD on study day 1 doses of 5, 10, 20, 40, 80, 160, 230 and 400 mg 2-(3,5-bis-trifluoromethyl-phenyl)-N-methyl-N-(6-morpholin-4-yl-4-o-tolyl-pyridin-3-yl)-isobutyramide were administered to the subjects orally as a drinking emulsion. At either 6 or 24 hours after the administration of the drug the subjects received a subcutaneous injection of 50 μg/kg of apomorphine in the lower part of the abdomen. The time of apomorphine administration was recorded. The subjects were brought into an upright sitting position immediately after the injection. They remained in this position until vomiting occurred or for at least 1 hour after the apomorphine injection. Vomiting is defined as regurgitation of approximately 25 ml or more of gastric contents. A retch is defined as a regurgitation producing less or no gastric content.

Nausea and/or vomiting was expected to occur on average within 10 minutes after the injection. The duration of nausea and/or vomiting after a subcutaneous dose of 50 μg/kg apomorphine was approximately 5 to 30 minutes. The number of vomits and retches were recorded.

The test groups were ranked in following order of “plasma concentration at the time of emesis test”. The plateau that was reached with the blockade had an average of 3 retches and vomits. If one assumes this as the point were efficacy is reached the test predicts that efficacious levels are reached at a concentration of 20 ng/ml plasma concentration.

The Spearman correlation test for the correlation between plasma concentration and the number of retches and vomits suggests a highly statistically significant relationship (p<0.01).

MAD

Subjects were dosed for 14 days with 2-(3,5-bis-trifluoromethyl-phenyl)-N-methyl-N-(6-morpholin-4-yl-4-o-tolyl-pyridin-3-yl)-isobutyramide. On day 14 the test was carried out as described in the SAD.

The MAD showed results that were consistent with the SAD.

Genotyping

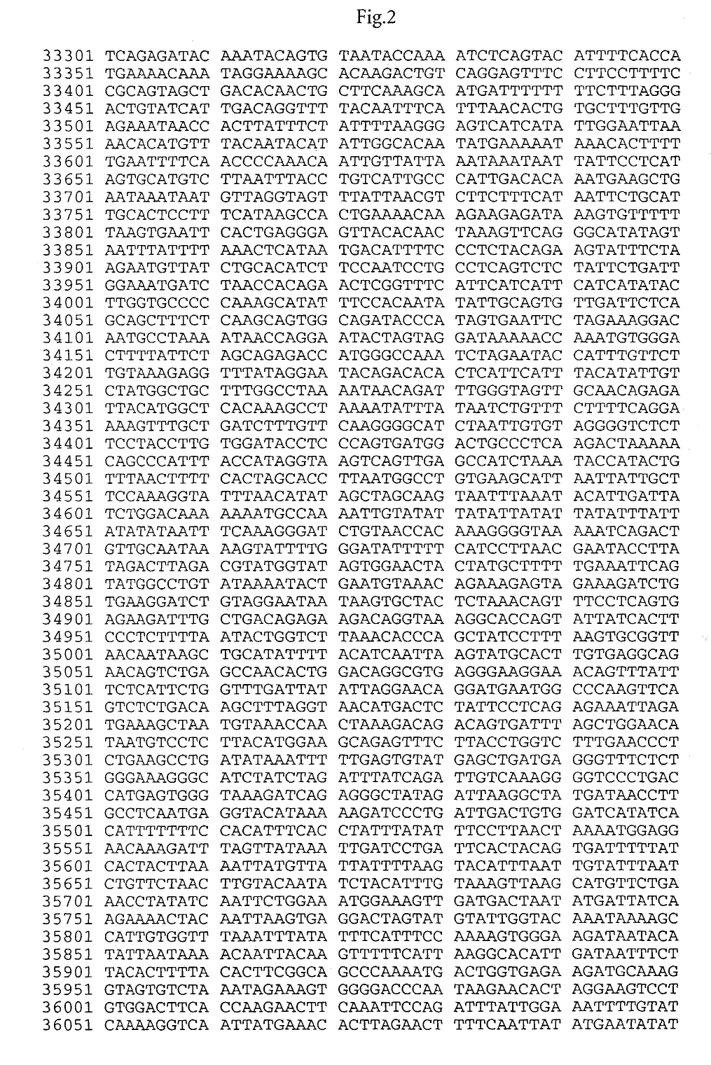

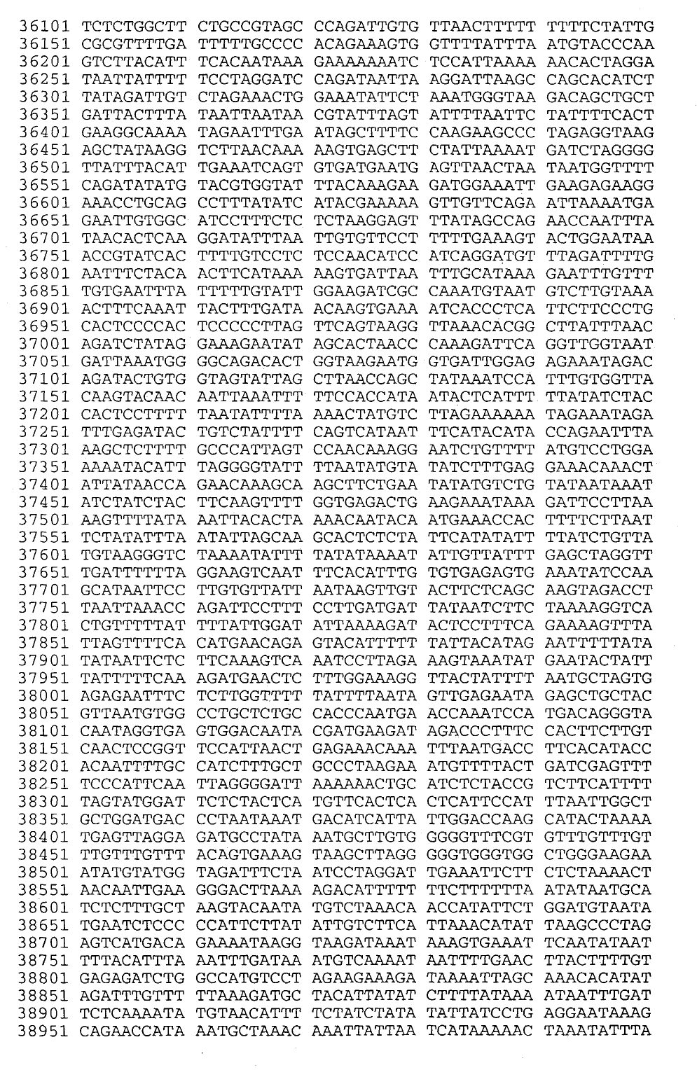

All participants of the SAD and MAD were tested for the single nucleotide polymorphism at position 41172 of the NKNA gene as defined by position in FIG. 2.

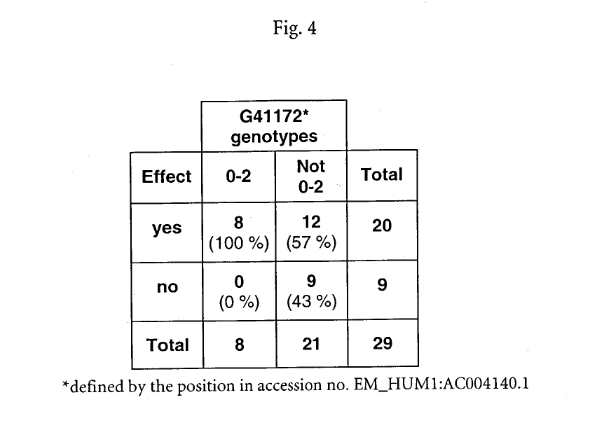

As the minimal concentration to achieve efficacy was found to be 20 ng/ml plasma concentration it was tested whether the single nucleotide polymorphism was found preferably in those individuals that had plasma concentration >20 ng/ml and who responded to the treatment, which is having ≦3 retches and vomits. The results are shown in FIG. 4.

Subjects containing the single nucleotide polymorphism G at position 41172 of the NKNA gene as defined by position in FIG. 2 in their genome were responding with a higher efficacy to the treatment as subjects not containing the single nucleotide polymorphism or those who are heterozygous only.

1. A method for correlating single nucleotide polymorphisms in the NKNA gene with the efficacy and compatibility of a pharmaceutically active compound administered to a human being which method comprises determining single nucleotide polymorphisms in the NKNA gene of a human being and determining the status of said human being to which a pharmaceutically active compound was administered by reference to polymorphism in the NKNA gene.

2. The method according to claim 1, wherein the nucleotide at at least one or more of the positions 33612, 33949,37025, 37114, 37434, 41112 and 41172 of the nucleotide sequence in FIG. 2 comprising the NKNA gene is determined.

3. The method according to claim 1, wherein the nucleotide at position 41172 of the NKNA gene as defined by the position of the nucleotide in FIG. 2 is determined.

4. The method according to claim 1, wherein the single nucleotide polymorphism in the NKNA gene is selected from at least one of
a C or a T at position 33612 in FIG. 2,
a T or a C at position 33949 in FIG. 2,
a C or a T at position 37025 in FIG. 2,
a G or an A at position 37114 in FIG. 2,
an A or a C at position 37434 in FIG. 2,
a T or a G at position 41112 in FIG. 2, and
a G or an A at position 41172 in FIG. 2, and combinations thereof as well as their reverse complement.

5. The method according to claim 1, wherein the pharmaceutically active compound is a NK-1 receptor antagonist.

6. The method according to claim 5, wherein the NK-1 receptor antagonist is a compound selected from the group consisting of 2-(3,5-bis-trifluoromethyl-phenyl)-N-methyl-N-(6-morpholin-4-yl-4-o-tolyl-pyridin-3-yl)-isobutyramide, 2-(3,5-bis-trifluoromethyl-phenyl)-N-[6-(1,1-dioxo-1λ⁶-thiomorpholin-4-yl)-4-o-tolyl-pyridin-3-yl]-N-methyl-isobutyramide, 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide; and a pharmaceutically acceptable acid addition salt thereof.

7. The method according to claim 5, wherein the NK-1 receptor antagonist is a compound selected from the group of NK-1 receptor antagonists under drug development designated GR205171, HSP-117, L 703,606, L 668,169, LY 303241, LY 306740, MK-869, R-544, Spantide III, WIN-62,577, GR 103,537, L 758,298, NKP608, CGP49823, CP-96,345, CP-99,994, CP-122,721, FK 888, GR203040, GR 82334, GR 94800, L 732,138, L 733,060, L 742,694, L 754,030, LY 303870, MEN 11149, PD 154075, RP-67580, RPR 100893, Spendide, Spantide II, SRI 40333, WIN-41,708, WIN-62,577, SR-48,968, L-659,877, MEN 10627, SR 144190, GR 94800, SR-142,801, R820, R486, SB 222200, L 758,298, NK-608, CGP 47899 and MEN 11467; or is a pharmaceutically acceptable acid addition salt thereof.

8. The method according to claim 5, wherein the NK-1 receptor antagonist is used for the treatment of a disease or a disorder selected from the group consisting of pain, headache, migraine, Alzheimer's disease, disorders of the central nervous system, depressive disorders, anxiety, emesis, psychosis, multiple sclerosis, attenuation of morphine withdrawal, cardiovascular changes, oedema, oedema caused by thermal injury, chronic inflammatory diseases, rheumatoid arthritis, asthma/bronchial hyperreactivity, allergic rhinitis, inflammatory diseases of the gut, ulcerative colitis, Crohn's disease, ocular injury and ocular inflammatory diseases, benign prostatic hyperplasia, motion sickness, treatment induced vomiting, cancer, malignant gliomas, and traumatic brain injury.

9. The method according to claim 5, wherein the NK-1 receptor antagonist is used for the treatment or prevention of disorders of the central nervous system, depressive disorders, anxiety, and emesis.

10. The method according to claim 1, wherein the step of determining single necleotide polymorphisms in the NKNA gene of the human being uses an assay comprising a differential nucleic acid analysis technique selected from the group consisting of direct mass-analysis of PCR products using mass spectrometry, direct analysis of invasive cleavage products, direct sequence analysis, allele specific amplification, allele specific hybridization, oligonucleotide ligation assay, Invader Assay, DNA chip analysis and restriction fragment length polymorphism.

11. A method for determining the efficacy and compatibility of a pharmaceutically active compound for a human being, which method comprises determining the presence of single nucleotide polymorphisms in the NKNA genomic sequence obtained from the human being which single nucleotide polymorphisms are correlated with the efficacy and compatibility of the pharmaceutically active compound, and thereby determining the efficacy and compatibility of the pharmaceutically active compound for the human being.

12. The method according to claim 11, wherein the nucleotide at at least one or more of the positions 33612, 33949, 37025,37114,37434,41112 and 41172 of the nucleotide sequence in FIG. 2 comprising the NKNA gene is determined.

13. The method according to claim 11, wherein the nucleotide at position 41172 of the NKNA gene as defined by the position of the nucleotide in FIG. 2 is determined.

14. The method according to claim 11, wherein the single nucleotide polymorphism in the NKNA gene is selected from at least one of
a C or a T at position 33612 in FIG. 2,
a T or a C at position 33949 in FIG. 2,
a C or a T at position 37025 in FIG. 2,
a G or an A at position 37114 in FIG. 2,
an A or a C at position 37434 in FIG. 2,
a T or a G at position 41112 in FIG. 2, and
a G or an A at position 41172 in FIG. 2, and combinations thereof as well as their reverse complement.

15. The method according to claim 11, wherein the pharmaceutically active compound is a NK-1 receptor antagonist.

16. The method according to claim 15, wherein the NK-1 receptor antagonist is a compound selected from the group consisting of 2-(3,5-bis-trifluoromethyl-phenyl)-N-methyl-N-(6-morpholin-4-yl-4-o-tolyl-pyridin-3-yl)-isobutyramide, 2-(3,5-bis-trifluoromethyl-phenyl)-N-[6-(1,1-dioxo-1λ⁶-thiomorpholin-4-yl)-4-o-tolyl-pyridin-3-yl]-N-methyl-isobutyramide, 2-(3,5-bis-trifluoromethyl-phenyl)-N-[6-(1,1-dioxo-1λ⁶-thiomorpholin-4-yl)-4-(4-fluoro-2-methyl-phenyl)-pyridin-3-yl]-N-methyl-isobutyramide; and a pharmaceutically acceptable acid addition salt thereof.

17. The method according to claim 15, wherein the NK-1 receptor antagonist is a compound selected from the group of NK-1 receptor antagonists under drug development designated GR205171, HSP-117, L 703,606, L 668,169, LY 303241, LY 306740, MK-869, R-544, Spantide III, WIN-62,577, GR 103,537, L 758,298, NKP608, CGP49823, CP-96,345, CP-99,994, CP-122,721, FK 888, GR203040, GR 82334, GR 94800, L 732,138, L 733,060, L 742,694, L 754,030, LY 303870, MEN 11149, PD 154075, RP-67580, RPR 100893, Spendide, Spantide 11, SR140333, WIN-41,708, WIN-62,577, SR-48,968, L-659,877, MEN 10627, SR 144190, GR 94800, SR-142,801, R820, R486, SB 222200, L 758,298, NK-608, CGP 47899 and MEN 11467; or is a pharmaceutically acceptable acid addition salt thereof.

18. The method according to claim 15, wherein the NK-1 receptor antagonist is used for the treatment of a disease or a disorder selected from the group consisting of pain, headache, migraine, Alzheimer's disease, disorders of the central nervous system, depressive disorders, anxiety, emesis, psychosis, multiple sclerosis, attenuation of morphine withdrawal, cardiovascular changes, oedema, oedema caused by thermal injury, chronic inflammatory diseases, rheumatoid arthritis, asthma/bronchial hyperreactivity, allergic rhinitis, inflammatory diseases of the gut, ulcerative colitis, Crohn's disease, ocular injury and ocular inflammatory diseases, benign prostatic hyperplasia, motion sickness, treatment induced vomiting, cancer, malignant gliomas, and traumatic brain injury.

19. The method according to claim 15, wherein the NK-1 receptor antagonist is used for the treatment or prevention of disorders of the central nervous system, depressive disorders, anxiety, and emesis.

20. The method according to any of claims 11, wherein the step of determining the presence of single nucleotide polymorphisms in the NKNA genomic sequence obtained from the human being uses an assay comprising a differential nucleic acid analysis technique selected from the group consisting of direct mass-analysis of PCR products using mass spectrometry, direct analysis of invasive cleavage products, direct sequence analysis, allele specific amplification, allele specific hybridization, oligonucleotide ligation assay, Invader Assay, DNA chip analysis and restriction fragment length polymorphism.

21. An isolated nucleic acid molecule selected from the group of:
the nucleic acid sequence of FIG. 2 with T at position 33612 as defined by the position in FIG. 2;
the nucleic acid sequence of FIG. 2 with C at position 33949 as defined by the position in FIG. 2;
the nucleic acid sequence of FIG. 2 with T at position 37025 as defined by the position in FIG. 2;
the nucleic acid sequence of FIG. 2 with A at position 37114 as defined by the position in FIG. 2;
the nucleic acid sequence of FIG. 2 with C at position 37434 as defined by the position in FIG. 2;
the nucleic acid sequence of FIG. 2 with G at position 41112 as defined by the position in FIG. 2;
the nucleic acid sequence of FIG. 2 with G at position 41172 as defined by the position in FIG. 2; or
a complementary strand thereof or a fragment thereof of at least 20 bases comprising at least one of the single nucleotide polymorphisms.

22. An allele-specific nucleic acid primer capable of detecting a polymorphism in the NKNA gene at one or more of the positions 33612, 33949, 37025, 37114, 37434, 41112 and 41172 of the nucleotide sequence in FIG. 2.

23. The nucleic acid primer according to claim 22, wherein the primer is selected from the group consisting of SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15 and SEQ ID NO. 16.

24. An oligonucleotide probe for detecting a polymorphism in the NKNA gene capable of hybridizing specifically to a nucleic acid wherein the nucleotide polymorphism in the NKNA gene is selected from at least one of
a C or a T at position 33612 in FIG. 2,
a T or a C at position 33949 in FIG. 2,
a C or a T at position 37025 in FIG. 2,
a G or an A at position 37114 in FIG. 2,
an A or a C at position 37434 in FIG. 2,
a T or a G at position 41112 in FIG. 2, and
a G or an A at position 41172 in FIG. 2, and combinations thereof as well as their reverse complement.

25. The oligonucleotide probe according to claim 24, wherein the oligonucleotide probe has a length of 17 to 50 nucleotides.

26. A diagnostic kit comprising at least one of an allele-specific nucleic acid primer capable of detecting a polymorphism in the NKNA gene at one or more of the positions 33612, 33949, 37025, 37114, 37434, 41112 and 41172 of the nucleotide sequence in FIG. 2 and an oligonucleotide probe for detecting a polymorphism in the NKNA gene capable of hybridizing specifically to a nucleic acid wherein the nucleotide polymorphism in the NKNA gene is selected from at least one of
a C or a T at position 33612 in FIG. 2,
a T or a C at position 33949 in FIG. 2,
a C or a T at position 37025 in FIG. 2,
a G or an A at position 37114 in FIG. 2,
an A or a C at position 37434 in FIG. 2,
a T or a G at position 41112 in FIG. 2, and
a G or an A at position 41172 in FIG. 2, and combinations thereof as well as their reverse complement.

27. A pharmaceutical pack comprising NK-1 receptor antagonists and instructions for administration of the drug to human beings tested for a single nucleotide polymorphism at one or more positions of the NKNA gene.

28. A pharmaceutical pack comprising NK-1 receptor antagonists and instructions for administration of the drug to human beings tested for a single nucleotide polymorphism at one or more of the positions 33612, 33949, 37025, 37114, 37434, 41112 and 41172 of the nucleotide sequence in FIG. 2.

29. Method for treating a NK-1 receptor ligand-mediated disease in a human being diagnosed as having a single nucleotide polymorphism at one or more of position 33612, 33949, 37025, 37114, 37434, 41112 and 41172 of the nucleotide sequence in FIG. 2 comprising the NKNA gene comprising administrating to a patient in need thereof a pharmaceutically effective amount of a NK-1 receptor antagonist.

30. A computer readable medium having stored thereon sequence information for the polymorphisms in the NKNA gene comprising polymorphisms in at least one of the following positions:
at position 33612 as defined by the position in FIG. 2,
at position 33949 as defined by the position in FIG. 2,
at position 37025 as defined by the position in FIG. 2,
at position 37114 as defined by the position in FIG. 2,
at position 37434 as defined by the position in FIG. 2,
at position 41112 as defined by the position in FIG. 2, and
at position 41172 as defined by the position in FIG. 2.

31. A method for performing sequence identification, said method comprising the steps of providing a nucleic acid sequence as claimed in claim 21 and comparing said nucleic acid sequence to at least one other nucleic acid sequence to determine identity.
